(19) United States
(12) Patent Application Publication
(10) Pub. No.: US 2004/0018247 A1
(43) Pub. Date: Jan. 29, 2004

(54) Formulation and the preservation process for animal and vegetable tissues

(76) Inventor: Walter Luis Wolmeister, Nova Petropolis (BR)

Correspondence Address: Clifford Kraft, 320 Robin Hill Dr., Naperville, IL, 60540, US

(21) Appl. No.: 10/208,516

(22) Filed: Jul. 29, 2002

(51) Int. Cl. G01N 1/30; G01N 33/48; A61K 33/24

(52) U.S. Cl. 424/655; 435/40.5

(57) ABSTRACT

FORMULATION AND THE RESPECTIVE PRESERVATION PROCESS OF ANIMAL AND VEGETABLE TISSUES, presented in liquid form and that has the particularity of having low basicity and a masking and complexing balanced system, where such characteristics grant to the product a high power of diffusion and distribution in the most internal regions of the hide structure, guaranteeing a fast transformation, and producing leathers with special properties, being able to be used in the processing of any type of hide, and being particularly indicated for bovine leathers of greater thickness (integral), where with the commonly used formulations a rather longer processing time is needed.

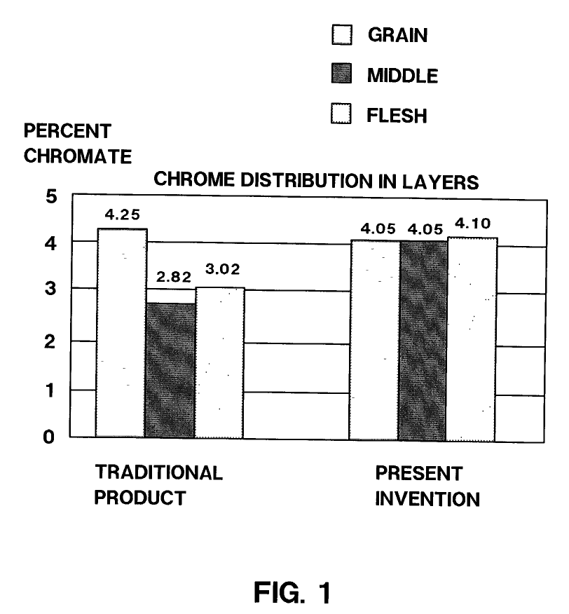

### FIELD OF THE INVENTION

The present invention relates to the field of tissue preservation and more particularly to a formulation and method of preserving animal and vegetable material such as tanning.

### SUMMARY OF THE INVENTION

The invention relates to a chrome formulation in liquid form and the respective process that has low basicity and a balanced masking and complexing system, where such characteristics grant to the product a high power of diffusion and distribution in the most internal regions of a hide structure, guaranteeing a fast preservation process and producing preserved tissues with special properties.

The proposed formulation can be used in the process of preserving any type of tissue, being particularly effective for bovine hides of greater thickness (integral), in which, with the commonly used formulations, a rather long process period is necessary. Therefore, the product's special characteristics allow the execution of an accelerated transformation process with other different advantages in relation to the preserved hides.

### STATE-OF-ART

Presently, the process of chrome transformation uses basic chrome sulphate with 33% basicity. The hides, after having been delimed and bated, are submitted to a salt-acid treatment called pickle. In this process salt (sodium chloride), sulphuric acid, and in many cases, formic acid is used. It is also possible in this step to execute an oxidation treatment for whitening the hides normally using sodium chlorite.

When the entire thickness of the hide is found with a pH around 2.5-2.8, the chromic formulation is added, which in these conditions can penetrate in the hides. The through-crossing of the chrome being complemented, an alkali is added, such as, for example, sodium bicarbonate or magnesium oxide, which raises system's pH to 3.7-3.9, when the tanning is completed and the leather is resistant to the temperature to 100° C. (Boiling Test).

As alternatives to this conventional process, we find in the market some products with the following characteristics:

Complexing agents. These are added to the pickle or together with the traditional chrome salts, with the purpose of decreasing the chrome reactivity with protein, increasing the penetration speed.

Carbonylic compounds. Modify the proteinaceous structure and provide higher chrome absorption.

There is modification of the process mechanism in both cases and not of the form of chrome spread and fixation, as occurs with the proposed formulation.

### PRINCIPAL ADVANTAGES OF THE DEVELOPED TANNING AGENT

1. High Speed of Crossing Through

Due to its low basicity, chrome penetrates in a fast and uniform form in the different areas of the hide, providing faster transformation than the processes with traditional chrome salts. Pickle can be left out and the tanning process can be shortened from 6 to 8 hours.

Table 1 in annex shows the average times of a transformation of full hides, comparing a traditional process and a process using the proposed formulation.

2. Excellent Chrome Absoption

This property can be observed through the excellent exhaustion of the chrome residual floats and the higher content of chrome fixated on leathers. In addition, the chrome distribution in layers is optimized. The graph in the annex shows the comparison between two processes, one with 6% Cromosal B (traditional chromic formulation with a concentration of 26% Cr₂O₃), and the other with 9.5% of the proposed formulation (with concentration of 15% Cr₂O₃).

It is observed that even with a smaller percentage of chrome oxide (in the example case, 1.56% of Cr₂O₃ for Cromosal and 1.42% Cr₂O₃ for the proposed formulation), the fixated chrome content is higher, being in average 3.56% Cr₂O₃ for Cromosal, and 4.07 Cr₂O₃ for the developed formulation. This positive effect of the product allows the total chrome percentage to be decreased, without loss on the chrome absorption by the hide.

### Chatacteristics of Leather Made with the Developed Formulation

Leathers tanned with the proposed formulation present, in crust, firm and fine grain, due to its low reactivity in the first steps of the process. Besides, positive effects were observed as to the filling and uniformness.

Physical-chemical properties are favored as well. Tables 2 and 3 in the annex show comparisons between the process with the developed formulation and the process with a traditional chrome salt.

Some different retannings, including of soft leathers, such as apparel and footwear nappa, were also made. Some advantages, such as greater softness and filling, were also observed.

Processes Versatility

The developed formulation can be used in different forms in the transformation process of hides into leathers. As a process that provides the highest advantages, we mention the so-called “Transformation Direct Process”.

In this process, the proposed formulation is added after the deliming step, eliminating the conventional pickle and the basification process. Chrome penetrates in the final deliming conditions, and the hide alkalinity itself is responsible for the basification.

This results in a saving of products and in eliminating hazardous and toxic products from the industrial environment (such as sulphuric acid and formic acid), and ease of the process control.

It is also possible, in accordance to the needs of each tannery, to perform a brief salt-acid treatment before adding the formulation, or even combining it with the traditional formulations. In this case, the process advantages are proportional to the amount of the proposed formulation used.

Characterization of the Formulation

Viewing the formulation, an object of the present invention is to be clearly understood and taken to practice through the proper technique in this technological sector. It will be clearly, precisely and objectively described, having as a basis its characterization using sufficient physical-chemical data to unequivocally individualize it.

Active ingredients: highly complexed chrome III salts.

Complexing agents: polymers of medium and low molecular weight (in the range of 500 to 10,000 units of atomic mass), formed with aromatic chains of 6 and 10 carbon atoms and saturated aliphatic chains, having hydroxyls (OH), amines (—NH2), carboxyls (—COOH), and sulphonics (—SO₃) functional groups.

FORMULATION OF THE SOLUTION: acid aqueous solution with Cr₂O₃ content from 12 to 18% and basicity from 10 to 20%.

The acid aqueous solution is prepared from the Chromium III sulfate acid solution with 12 to 18% of Cr₂O₃ content through an already known fabrication method by reducing the aqueous solution with from 20 to 60% sodium dichromate, using Sulfuric acid at 98% in quantities from 10 to 40% and sugars in aqueous solution as a reducing agent.

In the salt Chromium III aqueous solution, complexing agents are added in quantities from 5 to 20% synthesized from Vinylic acids, as Acrylic acid, Methacrylic, Maleic, Fumaric, in quantities from 1 to 10% Vinyl esters as Ethyl, Butyl, Hydroxyhexyl Acrylates and Methacrylates in quantities from 1 to 10%, Aromatic compounds such as Phenolsulfonate and Naphtalenesulfonate basis and condensed with Formaldehyde in quantities from 1 to 10%, Polyamides and Polyamines in quantities from 1 to 5%.

The Chromium III salt acid aqueous solution formulated with the complexants is adjusted to a basicity from 10 to 20% through addition of salt acids, as Sodium acid lactate, Sodium citrate, Sodium acid tartrate, and Sodium salicylate in the quantity from 1 to 8%.

### CHEMICAL GROUP TO WHICH THE COMPONENTS BELONG

ACTIVE: organic and inorganic Chrome III salts.

SOLVENT: water

PRODUCT IDENTITY: soluble in water, non-inflammable, non-volatile, corrosive.

DENSITY: between 1.35 and 1.70 g/ml.

PHYSICAL STATE: viscous liquid of dark green color.

pH IN 10% AQUEOUS SOLUTION: between 2.0 and 3.0.

TABLE 1
Comparison of the processing time (integral)

| | TRANS- | | | |
|---|---|---|---|---|
| | FORMATION | | FORMULA- | |
| | (TRADI- | | TION (PRO- | |
| PROCESS | TIONAL) | | POSED) | |
| Pickle | 5 | hours | 0 | |
| Tanning | 3 | hours | 12 | hours |
| Basifying | 12 | hours | 0 | |
| Total | 20 | hours | 12 | hours |

TABLE 2
Physical-mechanical results of semi-chrome (1.4-1.6 mm)

| | TRADITIONAL | PROPOSED |
|---|---|---|
| Tensile strength (N/mm²) | 11.3 | 12.0 |
| Elongation (5) | 37.6 | 48.3 |
| Tearing resistance (N/mm) | 47.8 | 51.0 |
| Lastometer (mm) | 8.3 | 9.7 |

TABLE 3
Physical-mechanical results of crust (1.2-1.4 mm)

| | TRADITIONAL | PROPOSED |
|---|---|---|
| Tensile strength (N/mm²) | 10.4 | 13.3 |
| Elongation (5) | 35.6 | 42.5 |
| Tearing resistance (N/mm) | 51.4 | 56.4 |
| Lastometer (mm) | 8.5 | 8.2 |

What is claimed is:

1. Formulation for preserving animal and vegetable tissues containing chrome III salts and complexing agents comprising: chromic acid in aqueous solution with a Cr₂O₃ content of around 12.0 to 18.0% and a sufficient quantity of a complexing agent containing polymers of molecular weight in the range of around 500 to 10,000, formed with aromatic chains of 6 and 10 carbon atoms and saturated aliphatic chains, having functionals chosen from the group containing hydroxyls (—OH), amines (—NH₂), carboxyls (—COOH), sulphonics (—SO₃H) to achieve a density between around 1.35 and 1.70 g/ml with acid salts added to achieve a final pH in aqueous solution of 10% concentration of between around 2.0 and around 3.0.

2. The formulation of claim 1 wherein said acid salts are chosen from the group containing sodium acid lactate, sodium citrate, sodium acid tartrate, and sodium salicylate.

3. A formulation for preserving animal and vegetable tissues containing essentially of: chromium (III) acid salts with around 10.0 to around 20.0% Cr₂O₃ content; complexing agents added in quantities from around 5 to 20% containing essentially acids chosen from the group containing vinylic acid, acrylic acid, methacrylic acid, maleic acid, and fumaric acid in quantities from around 1 to 10%; vinyl esters chosen from the group containing ethyl, butyl, hydroxyhexyl, acrylate and methacrylate esters in quantities from around 1 to 10%; aromatic compounds chosen from the group containing phenolsulfonate and naphtalenesulfonate in quantities from around 1 to 10%; and polyamides or polyamines in quantities from around 1 to 5% with PH being adjusted to around 2 to 3 in a 10% aqueous solution though addition of acidic salts chosen from the group containing sodium acid lactate, sodium citrate, sodium acid tartrate, and sodium salicylate.

4. The formulation of claim 3 wherein said complexing agents are condensed with formaldahyde.

5. A tanning formulation comtaining chrome III salts and complexing agents comprising: chromic acid in aqueous solution with a Cr₂O₃content of around 12.0 to 18.0% and a sufficient quantity of a complexing agent containing polymers of molecular weight in the range of around 500 to 10,000 to achieve a density between around 1.35 and 1.70 g/ml.

6. The tanning formulation of claim 5 with sufficient acidic salts added to achieve a final PH of around 2 to 3 in 10% aqueous solution.

7. The tanning formulation of claim 6 wherein said acidic salts are chosen from the group containing sodium acid lactate, sodium citrate, sodium acid tartrate and sodium silcylate.
